(19) United States
(12) Patent Application Publication
(10) Pub. No.: US 2004/0181244 A1
(43) Pub. Date: Sep. 16, 2004

(54) Vascular anastomosis device

(76) Inventor: Hoon-Bum Lee, Seoul (KR)

Correspondence Address: LADAS & PARRY, 5670 WILSHIRE BOULEVARD, SUITE 2100, LOS ANGELES, CA, 90036-5679, US

(21) Appl. No.: 10/478,764

(22) PCT Filed: May 31, 2002
(86) PCT No.: PCT/KR02/01038

(30) Foreign Application Priority Data
May 31, 2001 (KR) 2001-0030281

(51) Int. Cl. A61B 17/08

(52) U.S. Cl. 606/153

(57) ABSTRACT

The present invention provides a vascular anastomosis device of easily anastomosing the end of a blood vessel to the side of another blood vessel, which comprises a cylindrical body member having a longitudinally inner passege, a glanslike member positioned at the proximal end of the body member, the member having a larger diameter than the body member and having a plurality of hooking means which are formed along the outer circumferential surface of the distal portion thereof, and a sliding member installed on the outer surface of the body member.

### TECHNICAL FIELD

The present invention relates to a device of anastomosing blood vessels, more particularly, the device of easily anastomosing the end of a blood vessel to the side of another blood vessel.

### BACKGROUND ART

The lack of exercise and occidentalization of eating habits are factors of increasing the incidence rate of vascular diseases from year to year. In particular, most of vascular diseases are cardiac infarction and angina pectoris caused by the ischemia that a blood vessels is clogged feeding nutriment and oxygen to the heart.

In order to treat the clogged vessel or almost clogged vessel, the pharmaco-therapy using muscle relaxants or anti-calcium drugs for expansion of vessel muscle and the operative-therapy have been practiced. The pharmaco-therapy is useful for treatment at the initial symptom but is not so when the clogged procedure was progressed to the fair extent; therefore, the pharmaco-therapy is not substantial treatment and the operative-therapy has been generally practiced being the direct treatment of clogged vessel.

The operative-therapy is categorized into an internal operation and a surgical operation. The internal operation is to insert a stent into the clogged region (atresia region) through a blood vessel such as the femoral vessel and then expand the stent. The method of employing an expansible stent and apparatuses therefor are disclosed in U.S. Pat. No. 3,416,531. However, when a certain time elapses after the internal operation according to such method, lesion tissues protrude between nets of the stent and new granular tissues grow on the inner surface of stent, whereby the atresia phenomenon reoccurs.

Accordingly, as a further essential solution to this problem, the surgical operation has been carried out opening the heart and connecting the atresia vessel by a bypass with a vessel harvested from other organ of a patient (mainly, leg vein). In this surgical operation, one end of the vein is connected to IMA (Internal Mammary Artery: the artery feeding nutriment and oxygen to the organ and muscle of the chest and abdomen) by the end-to-end or end-to-side way, and the other end is connected to the vessel beyond the atresia region by the end-to-side or side-to-side way.

Meanwhile, a blood vessel comprises the intima, the media and the adventitia, and, in anastomosis of two vessels, the intima must be connected to the intima with these vessels everted. For anastomosis of severed vessels, and reconstruction by the skin flap transplantation, as well as treatment of cardiac diseases like the above, the specialist in the microsurgery secures a magnified operative visual-field with a microscope or a powerful magnifier and then sutures vessels or tissues one by one with a suture (a stitching fiber). The suture can be carried out only by the microsurgical specialist and thus is very time-consuming and heavy work. In particular, it is very difficult to suture one by one vessels being in the portion like the heart which pulsates periodically. Accordingly, it is necessary to stop the pulsation of heart for a long time (at least more than 3 hours) by causing the heart attack intentionally in operation.

Of these vascular anastomosis operations, the end-to-side type is very delicate and frequently required. Accordingly, in order to solve the problems of suture operation, various devices have been invented and some of them are explained as below.

U.S. Pat. No. 4,523,592 shows anastomotic coupling means capable of conducting both end-to-end and end-to-side anastomosis in which a pair of coupling disc members cooperate to couple the vessels, one of the members having spaced apart hook members and the other member having receptor cavities aligned with said hook members for locking the members together in a successful anastomatic procedure with tissue everted and secure on said hook members. However, because of the structural limitation, it is difficult to strongly lock the member having receptor on the side of vessel. Furthermore, two coupling disc members must be coupled to the vessels respectively and thereby the operation time becomes longer.

U.S. Pat. No. 5,752,966 shows end-to-end and end-to-side anastomotic devices respectively, and the end-to-side anastomotic device of them comprises a semi-rigid anastomotic ring and a harness assembly. In operation, the semi-rigid anastomotic ring is joined to the end portion of a vessel and the harness assembly is joined to the lateral portion of other vessel, then the semi-rigid anastomotic ring and the harness assembly are engaged. However, the harness assembly must be installed in the interior surface of the lateral portion of vessel, which requires the relatively large incision and may cause the possibility of bleeding after anastomosis. Also, this patent has a problem of long anastomosis time because two members are joined to each vessel, separately.

U.S. Pat. No. 6,206,913 shows anastomotic stents for connecting a graft vessel to a target vessel employing an outer flange and an inner flange, wherein the outer flange is joined to the end portion of a vessel and the inner flange is joined to the lateral portion of other vessel, then these are interlocked. However, this patent also has a problem of long anastomosis time because two members are joined to each vessel separately.

As mentioned above, these prior arts have the problem of long anastomosis time because two members are joined to each vessel separately and then engaged to each other. In the meantime, U.S. Pat. No. 4,366,819 shows an end-to-side anastomotic fitting requiring the joint procedure to only one vessel. This fitting which comprises a tube, a ringflange, a fixation ring and a locking ring is used in the procedure of joining the end portion of vessel to the tube with the ringflange, inserting them through the lateral portion of an incised vessel, and then clamping the fixation ring thereto. However, in view of its configuration, the joint between the vessel and the ringflage is not strong, and the joint between the vessel and the fixation ring also is not strong because the joint between them is achieved by engagement of the fixation ring to the lateral portion of vessel. Accordingly, despite the merit on not employing a separate member, this device has an essential problem that it is difficult to achieve the goal of anastomosis.

### SUMMARY OF INVENTION

The objects of the present invention are to solve the problems described above for once and all.

The first object of the present invention is to provide a device capable of remarkably reducing the operation time by conducing anastomosis in a series of procedures without joining constitutional elements to the end and lateral portions of two vessels, separately.

The second object of the present invention is to provide a device capable of making an end-to-side anastomosis stronger.

The third object of the present invention is to provide a device allowing the endoscopic operation through a small incision in the operative site.

In order to accomplish these objects, the vascular anastomotic device according the present invention for anastomosing the end portion of a vessel to the lateral portion of another vessel, comprises,

a cylindrical body member having a longitudinally inner passage;

a glans-like member positioned at the proximal end of the body member, the member having a larger diameter than the body member and having a plurality of hooking means which are formed along the outer circumferential surface of the distal portion thereof; and

a sliding member installed on the outer surface of the body member.

The directions described in the present specification can be illustrated referring to a 3-dimentional coordinates in FIG. 1 wherein “proximal direction” means +y direction and “distal direction” means -y direction. One vessel (A) is inserted into the cylindrical body member in an axial direction (y direction) and is then everted over the glans-like member to hook at the hooking means. At this state, another vessel (B) is positioned in xz plane being the perpendicular direction to y direction, and then a lateral plane thereof is incised, subsequently, the glans-like member is inserted through the incision into the vessel (B) to hook at the hooking means. The detailed anastomosis procedure will be illustrated later referring to drawings.

In an embodiment, the sliding member is configured to move readily backward and forward on the body member so that both vessels can be strongly engaged by moving the sliding member toward the glans-like member wherein the vessels have hooked at the hooking means of the glans-like member. In this case, the sliding member may have grooves on its proximal surface, into which the hooking means are inserted. In another embodiment, the body member may include a supporting part at its distal portion, the supporting part having a larger diameter than that of the body member, to prevent the sliding member from being taken off.

The body member, glans-like member and supporting part may be made in the form of separate parts and then assembled; or the body member and glans-like member or the body member and the supporting part may be made integratedly, in the case of integrated body member and glans-like member, the supporting part is assembled thereto, and in the case of integrated body member and supporting part, the glans-like member is assembled thereto. In an embodiment, the body member, and glans-like member and supporting part are made integratedly and the sliding member is assembled thereto.

In an embodiment, the body member comprises two or more parts so that it can be disassembled and assembled. For instance, the body member comprises a pair of semi-cylindrical, symmetrical units, each unit having interlocking means on the vertical surface (yz plane). Preferably, the body member comprises a pair of semi-cylindrical, symmetrical units wherein one side of both units has a hinge connecting them to each other and the other side of each unit has interlocking means. This configuration removes the cumbersome procedure of inserting the vessel (A) into the body member because it is possible to place a pair of semi-cylindrical, symmetrical units spread on the vessel (A) and then interlock them to complete the procedure, whereby the anastomosis operation can be conducted through a further small incision.

Since the glans-like member must be inserted through the incision into the vessel (B), preferably it is of an oval shape in view of the vertical plane (xz plane) and is curvilinearly canted. More preferably, the proximal portion of the glans-like member is leaned by about 5 to 30 degrees, so that it is easy to insert the glans-like member through the incision into the vessel (B).

The sliding member may also comprise two or more units capable of being disassembled and assembled and each unit has interlocking means on the vertical plane (yz plane). In a preferable embodiment, the sliding member comprises a pair of semi-cylindrical units, one side of both units having a hinge connecting themselves to each other and the other side of each unit having interlocking means.

In the configuration that the sliding member can move forward and backward, clamping the sliding member to the hooking means of the glans-like member can be classified to two manners: one is the case that the hooking means sticks in the sliding member and the other is the case that the hooking means is inserted into the groove formed on the proximal surface of the siding member to be clamped thereto. In the former case, the sliding member must be made of soft materials to the extent that the hooking means stick thereto, whereas in the latter case, the sliding member can be made of said soft materials or rigid materials. In the latter case, however, since the groove of the sliding member must clamp the hooking means to join thereto, a means for guiding the forward and backward movement of the sliding member should be formed on the interface between the body member and sliding member, to conform the positions of the groove and hooking means. However, in the case that the vertical sectional surface of the body member is of an oval shape and the corresponding inner surface of the sliding member is also of the oval shape, as in the glans-like member, said separate guiding means is not necessary.

In a preferable embodiment of the present invention, the body member, glans-like member and supporting part are made integratedly, and this integrated configuration comprises a pair of assembly units along the axial plane (yz plane), one side of both units being connected with a hinge and the other side of each unit having interlocking means, and the body member and glans-like member are of the oval shape in the vertical sectional view (in xy plane), the proximal portion of the glans-like member is curvilinearly canted in which the glans-like member comprises a pair of assembly units along the axial plane, one side of both units being connected with a hinge and the other side of each unit having interlocking means.

The interlocking means on both assembly units may have a diversity of configurations; however, in consideration of blood vessels being micro organs, the configuration is desirable in which the interlocking can be readily completed by applying a compressing force at one time. Therefore, the preferable configuration is that the interlocking means comprises a pair of male-female joints, the male joint being of an anchor shape (both arms of the anchor show off a spring effect) and the female joint being of a recess into which the anchor can be inserted.

The vascular anastomotic device must be made of biocompatible materials because it remains permanently in the human body. Such materials include titanium, stainless steel, rigid silicone, non-rigid silicone, etc. but are not limited to them.

The drawings are understood to be illustrative of the concepts disclosed herein to facilitate comprehension of the present invention. Further, the drawings are not to scale, and the scope of the present invention is not to be limited to the particular embodiments shown and described herein.

### DETAILED DESCRIPTION OF PREFERRED EMBODIMENTS

FIG. 1 shows a vascular anastomotic device according to one embodiment of the present invention in a perspective view, and FIG. 2A in a front view, FIG. 2B in a side view (at xz plane), respectively.

A vascular anastomotic device 100 generally comprises a cylindrical body member 200, a glans-like member 300, a supporting part 400 and a sliding member 500, in which the body member 200, glans-like member 300 and supporting part 400, being made integratedly, comprise a pair of symmetrical assembly units 110, 120 capable of being disassembled along an axial plane (yz plane). Each assembly unit 110, 120 has a pair of interlocking means on its vertical surface, one of them being a male joint 132 and the other being a female joint 134 into which the male joint 132 installed on the corresponding assembly unit 120 will be inserted.

The sliding member 500 moves forward and backward between the glans-like member 300 and the supporting part 400 along the outer surface of the body member 200, and has grooves 510 on its proximal surface for hooks 310 of the glans-like member 300 to be inserted into. The sliding member 500 also comprises a pair of assembly units 502, 504 being symmetrical on the axial plane (yz plane). In the case that the sliding member 500 is made of soft materials such as silicone, the hooks 310 of the glans-like member 300 can pierce into the proximal surface of such sliding member 500 as the sliding member 500 moves forward; therefore, the grooves 510 is not an indispensable element for the present invention.

The glans-like member 300 has a plurality of hooks 310 on its distal surface 302, and the proximal portion 304 is leaned and the lateral surface 306 is curvilinearly canted. Accordingly, the glans-like member 300 of such configuration can be inserted through a small incision in a vessel (B).

The supporting part 400 serves as preventing the sliding member 500 from being taken off backward but is not an indispensable element.

The cylindrical body member 200 and glans-like member 300 are of an oval shape in a cross-sectional view perpendicular to the axis (in xz plane). Further, the inner major diameter of the body member 200 being of the oval sectional shape is larger than the outer diameter of a vessel (A), whereas the inner minor diameter is shorter than the outer diameter. As a result, when the vessel (A) is engaged with the assembly units 100, 200, the vessel having a circular cross-section is rather pressed to be converted to an elliptical shape. In the cross-sectional view, the outer surface of the body member 200 is of the elliptical shape and the inner surface of the sliding member 500 corresponding thereto is also of the elliptical shape, whereby the hooks 310 of the glans-like member 300 can conform with the grooves 510 of the sliding member 500 without aid of a separate guiding means.

FIGS. 3A to 3F and FIGS. 4A and 4B shows a series of procedures of anastomosing two blood vessels in an end-to-side manner.

Referring to FIG. 3A, when the flow of blood is the same as shown in the drawing, a blocking device 600 is installed at the rear of a vessel (A) to block the blood flow, and then the vessel (A) is severed at the site to be anastomosed. Thereafter, a vascular anastomotic device 100 of the present invention is joined to the severed vessel (A) in which assembly units 110, 120 comprising a body member 200, a glans-like member 300 and a supporting part 400 are interlocked and assembly units 502, 504 of a sliding member 500 are then interlocked above the interlocked assembly units 110, 120. Although not illustrated for simplicity of the drawing, the rear of vessel (A) is expanded by the blood pressure caused by the blocking device 600. This phenomenon also takes place at a vessel (B) as the blocking device 600 is installed on the vessel (B), as mentioned later, which will not be illustrated in the drawing for the same reason.

In the next step, referring to FIG. 3B, the proximal portion of the vessel (A) is everted and then fixed on hooks 310 of a glans-like member 300, thereby the intima of vessel (A) being opened. As mentioned above, since the intima of a vessel must meet with the intima of other vessel for anastomosis, both vessels need to be everted. The proximal portion of vessel (A) is fixed on the hooks of the glans-like member 300 so that the vessel (A) will not be separated from the device 100 at the thereafter procedure.

In the next step, referring to FIG. 3C, when the flow direction of blood is the same as in the drawing, the blocking device 600 is installed at the rear of vessel (B) to block the flow of blood and then the lateral surface of vessel (B) is incised to make an incision 700. The length of incision 700 is desirable to be smaller than the outer diameter of glans-like member 300 (diameter of the largest portion). This is because the glans-like member 300 having a relatively large diameter can be inserted through the small diameter incision 700 due to the elasticity of vessel, and the small incision makes the possibility of bleeding less after anastomosis.

In the next step, referring to FIG. 3D, the anastomotic device 100 is inserted through the incision into the vessel (B), which can be readily conducted by first inserting the portion (a) of large width into the incision 700 and then the portion (b) of small width because the proximal portion of glans-like member 300 is leaned.

In the next step, referring to FIG. 3E, the tissue of vessel (B) is fixed on the hooks 310 of the glans-like member 300 by pushing the anastomosis device 100 forward. Because the vessel (A) was already fixed on the hooks 310, the anastomosis device 100 must be pushed to the extent that the hooks 310 pierce the vessel (B) to protrude therefrom.

In the next step, referring to FIG. 3F, the hooks 310 of the glans-like member 300 is joined to grooves (not shown) of a sliding member 500 by pushing the sliding member forward. The glans-like member 300 and sliding member 500 are strongly engaged to each other by the joint of the hooks 310 and grooves so that the vessels (A), (B) are clamped between them. Moreover, the vessel (A) is fixed over the glans-like member 300 with its intima opened so that the intima of vessel (B) meets only the intima of vessel (A). After completion of joint, the blocking devices 600 installed on the rear of vessels (A), (B) are removed, thereby the vascular anastomosis being finished.

FIGS. 4A and 4B show the procedure of rendering the anastomosed site further stably by using a resilient ring, which is another embodiment of the present invention. An engaging ring 800 is placed on a sliding member 500′ after an anastomotic device 100 is joined to a vessel (A), that is, is used at the step of FIG. 3A. The engaging ring 800 can take the place of the hooks 360 and grooves 510 being joined so that, in this case, a sliding member 500 of the anastomotic device 10′ may is fixed on a body member 200 without forward/backward movement and a separate supporting part 400 is not required and a sliding member 500′ may be configured not to have any grooves on its proximal surface. In another embodiment, the engaging ring 800, as an adjunctive element in the anastomotic device 100 of FIG. 1, may work as rendering the anastomosis of both vessels further strongly as used at the step of FIG. 3F.

Referring to FIG. 4A, the anastomotic device 100 in which the engaging ring 800 is installed on the sliding member 500′ is pulled from the inside of a vessel (B). Referring to FIG. 4B, the engaging ring 800 moves forward to make the anastomosed site stronger. The engaging ring 800 may be made of resilient materials such as a resilient silicone so that it shrinks at the step of FIG. 4B.

The present invention being thus described, it will be obvious that the same may be varied in many ways. Such variations are not to be regarded as a departure from the spirit and scope of the invention and all such modifications would be obvious to one skilled in the art.

### BRIEF DESCRIPTION OF THE DRAWINGS

FIG. 1 is a perspective view of a vascular anastomotic device according to one embodiment of the present invention.

FIGS. 2A and 2B are a front view and a side view of the device of FIG. 1, respectively.

FIGS. 3A to 3F are perspective views of a series of procedures of anastomosing two blood vessels in the end-to-side type employing the device of FIG. 1.

FIGS. 4A and 4B are perspective views of partial procedures of anastomosing two blood vessels employing another device of the present invention.

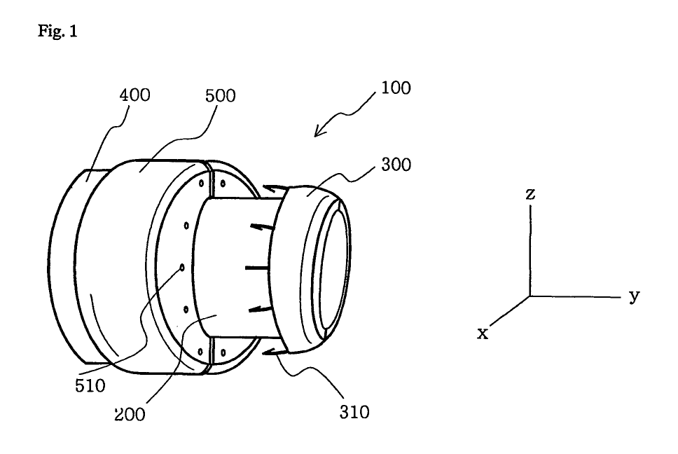

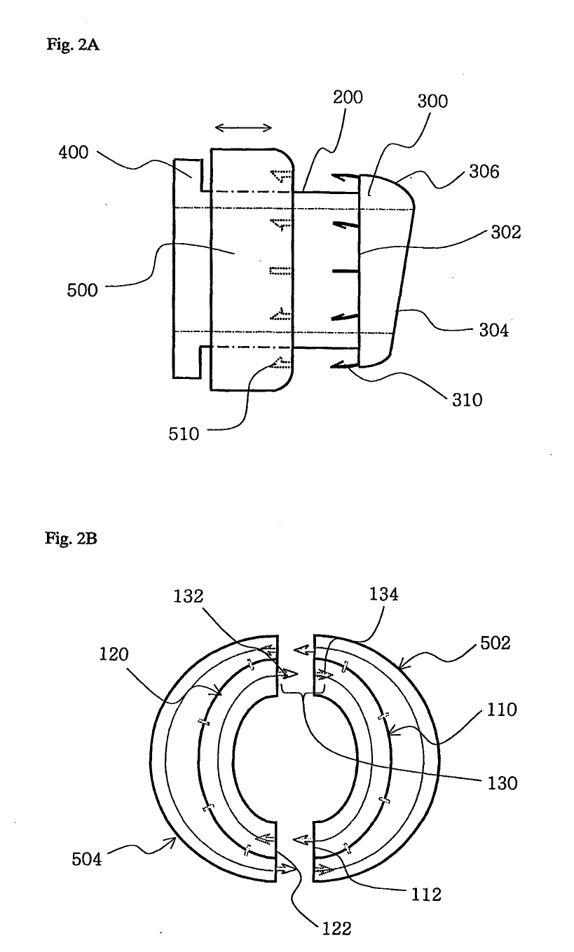

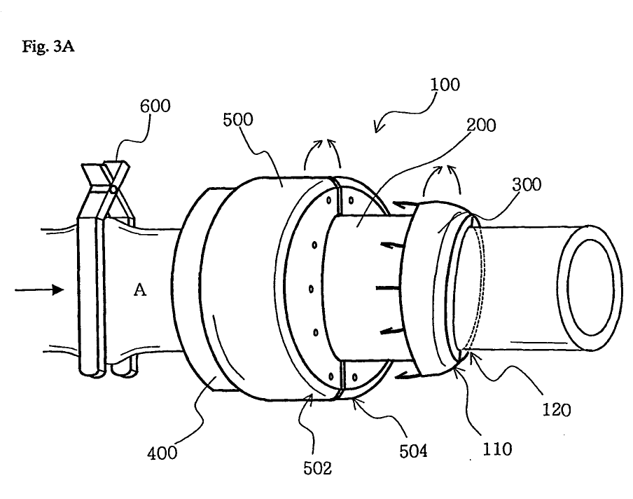

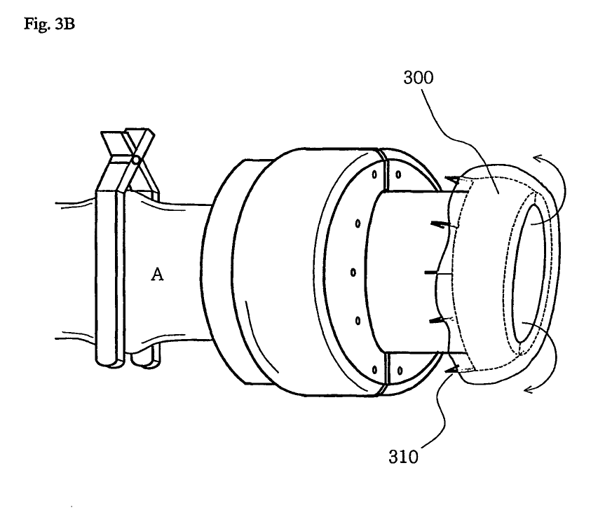

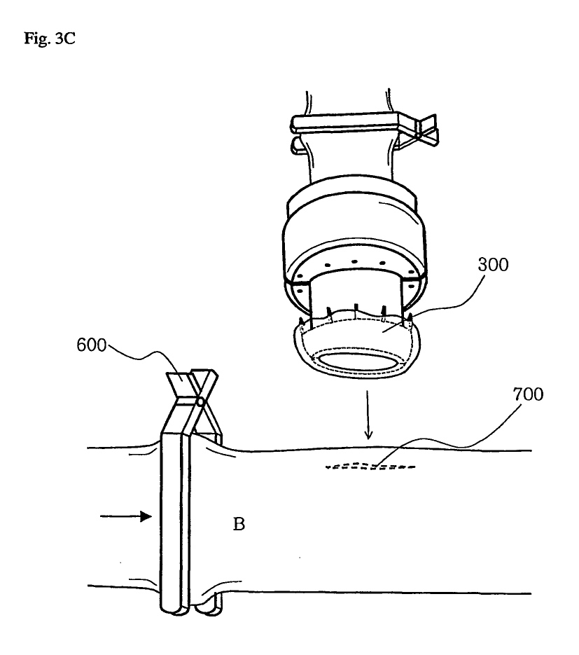

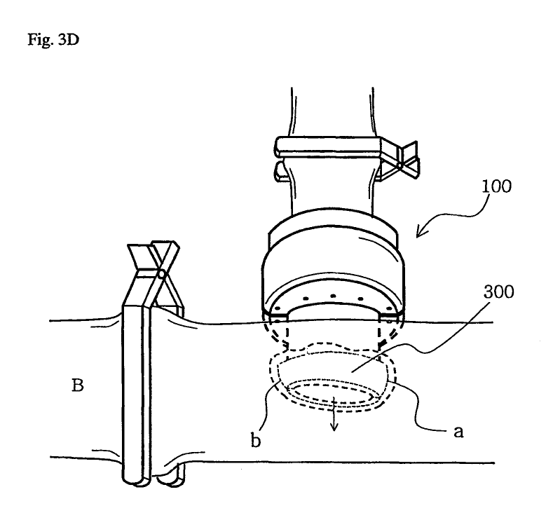

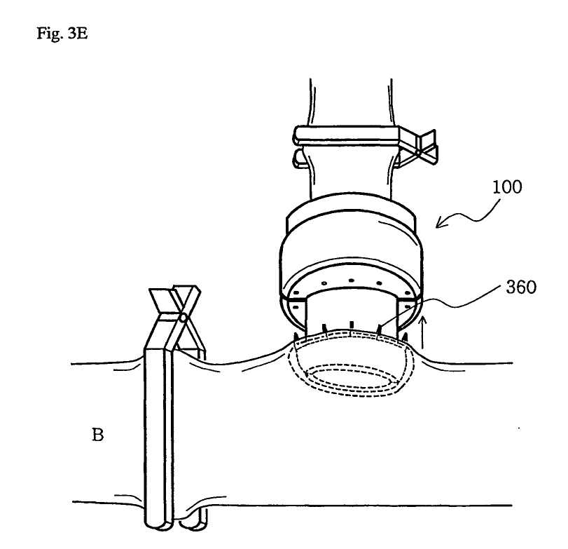

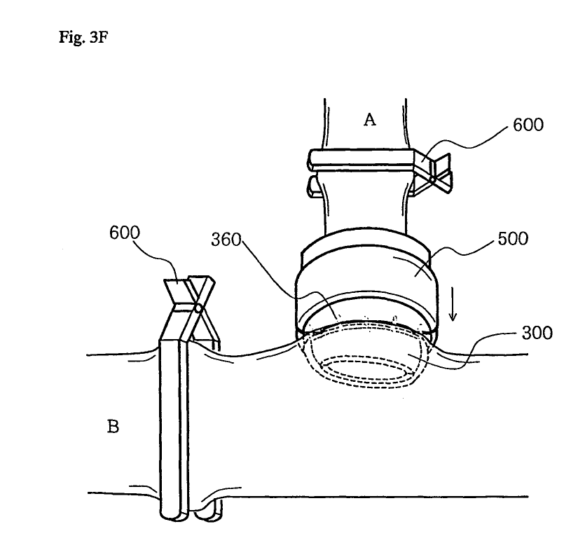

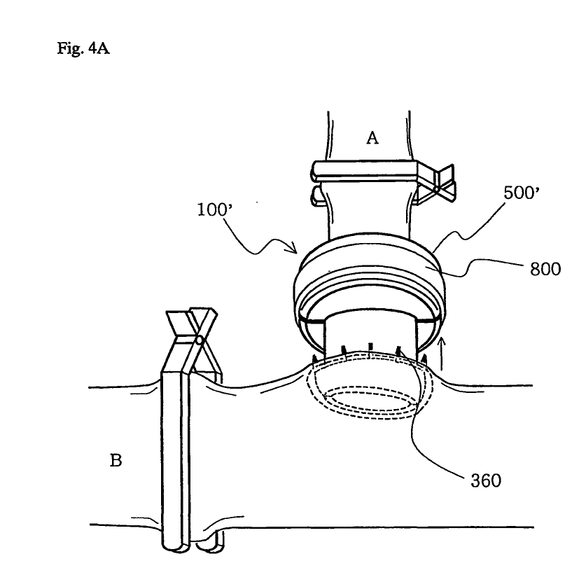

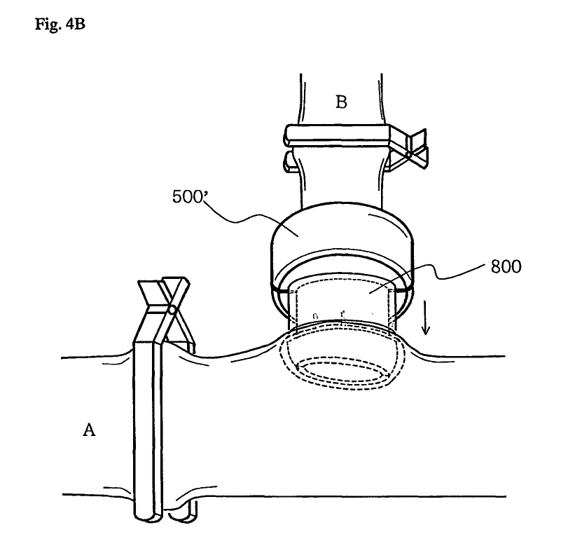

### DESIGNATION OF THE REFERENCE NUMBERS

100: vascular anastomotic device

200: body member

300: glans-like member

400: supporting part

500: sliding member

600: blocking device

700: incision

800: engaging ring

### Industrial Applicability

The vascular anastomotic devices of the present invention, being devices of anastomosing two blood vessels in the end-to-side type, need not engage two vessels to each elements of the device, separately, so that the anastomotic procedure is simple and the joint of anastomosed site is strong. Furthermore, it is not necessary to open the operational site fully but the anastomosis is possible through a small incision, which can reduce the operational time and relieve a patient's pain remarkably.

What is claimed is:

1. A vascular anastomotic device for anastomosing the end portion of a vessel to the lateral portion of another vessel, comprising,
a cylindrical body member having a longitudinally inner passage;
a glans-like member positioned at the proximal end of the body member, the member having a larger diameter than the body member and having a plurality of hooking means which are formed along the outer circumferential surface of the distal portion thereof; and
a sliding member installed on the outer surface of the body member.

2. The vascular anastomotic device according to claim 1, wherein the sliding member is configured to move readily backward and forward on the body member so that both vessels can be strongly engaged by moving the sliding member toward the glans-like member wherein the vessels have been fixed on the hooking means of the glans-like member.

3. The vascular anastomotic device according to claim 2, wherein the sliding member has grooves on its proximal surface, into which the hooking means are inserted.

4. The vascular anastomotic device according to claim 2 or 3, wherein the body member includes a supporting part at its distal portion, the supporting part having a larger diameter than that of the body member, to prevent the sliding member from being taken off.

5. The vascular anastomotic device according to claim 1 or 2, wherein the body member and sliding member comprise a pair of semi-cylindrical, symmetrical units, each unit having interlocking means on the vertical surface (yz plane).

6. The vascular anastomotic device according to claim 1 or 2, wherein the body member and sliding member comprise a pair of semi-cylindrical, symmetrical units in where one side of both units has a hinge connecting them to each other and the other side of each unit has interlocking means.

7. The vascular anastomotic device according to claim 1 or 2, wherein the glans-like member is of an oval shape in view of the vertical plane (xz plane) and is curvilinearly canted, and the proximal portion of the glans-like member is leaned by about 5 to 30 degrees.

8. The vascular anastomotic device according to claim 4, wherein the body member, glans-like member and supporting part are made integratedly, and this integrated configuration comprises a pair of assembly units along the axial plane (yz plane), one side of both units being connected with a hinge and the other side of each unit having interlocking means, and the body member and glans-like member are of the oval shape in the vertical sectional view (in xy plane), the proximal portion of the glans-like member is curvilinearly canted in which the glans-like member comprises a pair of assembly units along the axial plane, one side of both units being connected with a hinge and the other side of each unit having interlocking means.

9. The vascular anastomotic device according to claim 1, wherein the device further includes a resilient engaging ring which is placed on a sliding member in anastomosis operation and then move toward the glans-like member to make the anastomosed site stronger.
